SEQUENCE LISTING (2 sequences)

SEQ ID NO: 1 - protein, 327 aa, synthetic construct
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 577 aa, synthetic construct
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0217816 A1
(43) Pub. Date: Jul. 30, 2026

(54) METHODS OF TREATING HIDRADENITIS SUPPURATIVA

(71) Applicants: AbbVie Inc., North Chicago, IL (US); AbbVie Deutschland GmbH & Co. KG, Wiesbaden (DE)

(72) Inventors: Mona Akbari, Needham, MA (US); David A. Williams, Lake Forest, IL (US); Yinuo Pang, Lincolnshire, IL (US); Xiaohong Huang, Vernon Hills, IL (US); Tianyu Zhan, Lincolnshire, IL (US); Ziqian Geng, Chicago, IL (US); Yu Han, North Chicago, IL (US); Insa Winzenborg, Ludwigshafen (DE)

(21) Appl. No.: 19/147,659

(22) PCT Filed: Jan. 2, 2025
(86) PCT No.: PCT/US2025/010066

§ 371 Date: Jul. 11, 2025

Related U.S. Application Data
(60) Provisional application No. 63/617,327, filed on Jan. 3, 2024.
(60) Provisional application No. 63/562,501, filed on Mar. 7, 2024.
(60) Provisional application No. 63/698,216, filed on Sep. 24, 2024.

(51) Int. Cl. C07K 16/24; A61K 9/00; A61K 38/00; A61P 17/10

(52) U.S. Cl. CPC C07K 16/245; A61K 9/0019; A61K 38/00; A61P 17/10; C07K 2317/31; C07K 2317/64; C07K 2317/76

(57) ABSTRACT

The present disclosure relates to methods of treating hidradenitis suppurativa in a subject comprising administering to the subject a therapeutically effective amount of lutikizumab.

### CROSS-REFERENCE TO RELATED APPLICATIONS

This application claims the benefit of U.S. Provisional Application No. 63/617,327, filed Jan. 3, 2024, U.S. Provisional Application No. 63/562,501, filed Mar. 7, 2024, and U.S. Provisional Application No. 63/698,216, filed Sep. 24, 2024, the contents of each of which are hereby incorporated by reference in their entirety.

### REFERENCE TO A SEQUENCE LISTING XML

This application contains a Sequence Listing which has been submitted electronically in XML format. The Sequence Listing XML is incorporated herein by reference. Said XML file, created on Dec. 19, 2024, is named AVR-81825_SL.xml and is 3,652 bytes in size.

### BACKGROUND

Hidradenitis suppurativa (HS) is a debilitating inflammatory skin disease with a characteristic clinical presentation of recurrent or chronic painful, suppurating lesions (i.e., deep-seated nodules, abscesses, and sinuses) and scars that most commonly present in the apocrine gland-bearing areas or the intertriginous areas (e.g., axillary, inframammary, inguinal, and anogenital regions) of the body. The lesions are often malodorous, have purulent discharge, and may result in substantial disability and social stigma for patients and have a profound impact on their quality of life. Depression, anxiety, and an increased suicide risk have been reported in patients with HS, and the disease often has an adverse influence on a patient's sexual health. HS is often associated with comorbidities, such as obesity, metabolic syndrome, diabetes mellitus, inflammatory arthritis, polycystic ovary syndrome, and Crohn's disease.

The estimated world-wide prevalence of HS varies between <1% and 4%. There is often a significant delay (7.2 years on average) in establishing the diagnosis of HS after its initial presentation. Moderate to severe HS accounts for approximately 40% of patients who are newly diagnosed with HS.

There are no known cures or preventative measures for HS. As described in both the North American and European Clinical Management Guidelines for HS, treatment options include medical interventions such as topical therapies (e.g., exfoliants and peels), topical antibiotics (e.g., clindamycin), systemic antibiotics (e.g., clindamycin, tetracycline, rifampicin), anti-inflammatory therapies (e.g., corticosteroids, dapsone, ciclosporin A), hormones (e.g., antiandrogens and estrogens), retinoids (e.g., isotretinoin, acitretin), biologics (e.g., adalimumab, infliximab), analgesics (e.g., nonsteroidal anti-inflammatory drugs, opioids), and surgical treatments (e.g., deroofing, excision, laser). Except for adalimumab and secukinumab, these medical treatments are not authorized for the treatment of HS, and most have not been adequately studied. Thus, there is a high unmet need for new safe and efficacious HS therapies.

### SUMMARY

The present disclosure relates to methods of treating hidradenitis suppurativa in a subject comprising administering to the subject a therapeutically effective amount of lutikizumab. The methods described herein provide an effective and safe dosing regimen for administering lutikizumab to treat hidradenitis suppurativa in a subject.

Accordingly, in one aspect, the present disclosure relates to a method of treating hidradenitis suppurativa in a subject comprising administering to the subject a therapeutically effective amount of lutikizumab, wherein the therapeutically effective amount of lutikizumab comprises a first dose and a second dose of lutikizumab, wherein the first dose comprises about 300 mg to about 600 mg (e.g., about 300 mg, about 400 mg, about 500 mg, or about 600 mg) of lutikizumab, and the second dose comprises about 300 mg of lutikizumab.

### BRIEF DESCRIPTION OF THE DRAWINGS

FIG. 1 is a schematic diagram describing the Phase II M20-262 study. Subjects were randomized into four treatment groups. Subject dosing was every week with the final dose at Week 15. BL=Baseline; EOW=Every other week; EW=Every week; PBO=Placebo; SC=Subcutaneous; W=Week.

FIG. 2 shows the response rates and 95% confidence intervals (CIs) for HiSCR 50 at each visit (NRI-MI).

FIG. 3 shows the response rates and 95% confidence intervals (CIs) for NRS30 among Baseline NRS≥3 at each visit (NRI-MI).

FIG. 4 shows the response rates and 95% confidence intervals (CIs) for HiSCR 75 at each visit (NRI-MI).

FIG. 5 shows the response rates and 95% confidence intervals (CIs) for HiSCR 90 at each visit (NRI-MI).

FIG. 6 shows the adjusted response rates and 95% confidence intervals (CIs) for HiSCR 50 at each visit.

FIG. 7 shows the adjusted response rates and 95% confidence intervals (CIs) for HiSCR 75 at each visit.

FIG. 8 shows the adjusted response rates and 95% confidence intervals (CIs) for HiSCR 90 at each visit.

FIG. 9 is a schematic diagram describing the Phase III M20-465 study. BL=baseline; EOW=every other week; EW=every week; HCP=healthcare provider; LTE=long-term extension. * Note: At BL and Weeks 1, 2, 4, 6, 8, 12, 16-21, 23, 25, 27, 29, 31, 32, 33, 35, 37, 39, 41, 43, 45, 47, 49, 51, dose administration is given by designated HCP. At Weeks 3, 5, 7, 9, 10, 11, 13, 14, 15, 22, 24, 26, 28, 30, 34, 36, 38, 40, 42, 44, 46, 48, and 50, dose administrations are self-injections by subject. * Week 52 is only an end-of-study site visit, no dose is given to subjects at this visit.

FIG. 10 shows predicted pharmacokinetics (PK) profiles for 300 mg every week (EW) with or without a loading dose of 600 mg.

FIG. 11 shows achievement of ISH4-55, IHS4-75, and IHS4-90 at Week 16. CI, confidence interval; EOW, every other week; EW, every week; IHS4, International Hidradenitis Suppurativa Severity Score System; Luti, lutikizumab; NRI, non-responder imputation; NRI-MI, non-responder imputation incorporating multiple imputation. IHS4 55/75/90 were defined as at least a 55%, 75%, or 90% reduction, respectively, from baseline in IHS4 [Number of nodules (inflammatory)×1]+[Number of abscesses×2]+[Number of draining tunnels×4]. NRI-MI was used to handle missing data for IHS4-55, and NRI was used for IHS4-75/90.

### DETAILED DESCRIPTION

The present disclosure describes the unexpected discovery that administration to a human subject afflicted with moderate to severe hidradenitis suppurativa of a first dose of about 300 mg to about 600 mg (e.g., about 300 mg, about 400 mg, about 500 mg, or about 600 mg) of lutikizumab and a second dose of about 300 mg of lutikizumab induces clinical responses.

As disclosed herein, the present disclosure relates to the following embodiments.

Embodiment 1. A method of treating hidradenitis suppurativa (HS) in a subject comprising administering to the subject a therapeutically effective amount of lutikizumab, wherein the therapeutically effective amount of lutikizumab comprises a first dose and a second dose of lutikizumab, wherein the first dose comprises about 300 mg to about 600 mg of lutikizumab, and the second dose comprises about 300 mg of lutikizumab.

Embodiment 2. The method of embodiment 1, wherein the subject is a patient afflicted with moderate to severe HS.

Embodiment 3. The method of embodiment 1 or 2, wherein the subject is an adult patient or an adolescent patient.

Embodiment 4. The method of any one of embodiments 1-3, wherein the subject has a total abscess and inflammatory nodule (AN) count of ≥5 and has HS lesions present in at least 2 distinct anatomic areas at baseline.

Embodiment 5. The method of any one of embodiments 1-4, wherein the subject is naïve to a biologic therapy for HS.

Embodiment 6. The method of any one of embodiments 1-4, wherein the subject has been treated with a prior biologic therapy for HS and had intolerance or an inadequate response to the therapy.

Embodiment 7. The method of embodiment 6, wherein the prior biologic therapy comprises an anti-TNF treatment for HS.

Embodiment 8. The method of any one of embodiments 1-7, wherein the first dose comprises about 300 mg to about 500 mg, about 300 mg to about 400 mg, about 400 mg to about 600 mg, about 400 mg to about 500 mg, or about 500 mg to about 600 mg of lutikizumab.

Embodiment 9. The method of any one of embodiments 1-7, wherein the first dose comprises about 300 mg, about 400 mg, about 500 mg, or about 600 mg of lutikizumab.

Embodiment 10. The method of any one of embodiments 1-9, wherein the first dose is administered to the subject at Week 0 (Day 1).

Embodiment 11. The method of any one of embodiments 1-10, wherein the second dose is administered to the subject at Week 1 (Day 8).

Embodiment 12. The method of any one of embodiments 1-11, wherein the therapeutically effective amount of lutikizumab comprises administration of the first dose of lutikizumab at Week 0 (Day 1) and the second dose of lutikizumab at Week 1 (Day 8).

Embodiment 13. The method of any one of embodiments 1-12, wherein the therapeutically effective amount of lutikizumab comprises administration of the first dose of lutikizumab at Week 0 (Day 1), the second dose of lutikizumab at Week 1 (Day 8), and about 300 mg of lutikizumab at Week 2 (Day 15) and every week thereafter.

Embodiment 14. The method of any one of embodiments 1-12, wherein the therapeutically effective amount of lutikizumab comprises administration of the first dose of lutikizumab at Week 0 (Day 1), the second dose of lutikizumab at Week 1 (Day 8), and about 300 mg of lutikizumab at Week 2 (Day 15) or Week 3 (Day 22) and every other week thereafter.

Embodiment 15. The method of any one of embodiments 1-12, wherein the therapeutically effective amount of lutikizumab comprises administration of the first dose of lutikizumab at Week 0 (Day 1), the second dose of lutikizumab at Week 1 (Day 8), about 300 mg of lutikizumab every week starting Week 2 (Day 15) for a period of time, and after the period of time, administration of about 300 mg of lutikizumab every other week.

Embodiment 16. The method of any one of embodiments 1-12, wherein the therapeutically effective amount of lutikizumab comprises administration of the first dose of lutikizumab at Week 0 (Day 1), the second dose of lutikizumab at Week 1 (Day 8), about 300 mg of lutikizumab every other week starting Week 2 (Day 15) for a period of time, and after the period of time, administration of about 300 mg of lutikizumab every week.

Embodiment 17. The method of any one of embodiments 1-16, wherein the first dose comprises about 300 mg of lutikizumab.

Embodiment 18. The method of embodiment 17, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every week thereafter.

Embodiment 19. The method of embodiment 17, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every other week thereafter.

Embodiment 20. The method of embodiment 17, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every week starting Week 2 (Day 15) for a period of time; and (d) after the period of time, administration of about 300 mg of lutikizumab to the subject every other week.

Embodiment 21. The method of embodiment 17, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every other week starting Week 2 (Day 15) for a period of time; and (d) after the period of time, administration of about 300 mg of lutikizumab to the subject every week.

Embodiment 22. The method of any one of embodiments 1-16, wherein the first dose comprises about 600 mg of lutikizumab.

Embodiment 23. The method of embodiment 22, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every week thereafter.

Embodiment 24. The method of embodiment 22, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) or Week 3 (Day 22) and every other week thereafter.

Embodiment 25. The method of embodiment 22, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every week starting Week 2 (Day 15) for a period of time; and (d) after the period of time, administration of about 300 mg of lutikizumab to the subject every other week.

Embodiment 26. The method of embodiment 22, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every other week starting Week 2 (Day 15) or Week 3 (Day 22) for a period of time; and (d) after the period of time, administration of about 300 mg of lutikizumab to the subject every week.

Embodiment 27. The method of any one of embodiments 1-26, wherein the lutikizumab is administered by subcutaneous injection.

Embodiment 28. The method of any one of embodiments 1-27, wherein the subject achieves one or more of the endpoints selected from the group consisting of: (1) HiSCR 50; (2) Numeric Rating Scale (NRS) 30; (3) HiSCR 75; (4) HiSCR 90; (5) >1.5 reduction from baseline in draining fistula count; (6) >21.3% reduction from baseline in draining fistula count; (7) no increase in draining tunnels (dTs) from baseline; (8) complete elimination of dTs; (9) IHS4-55; (10) IHS4-75; and (11) IHS4-90.

Embodiment 29. A method of treating moderate to severe hidradenitis suppurativa (HS) in a patient, wherein the patient has had an inadequate response or intolerance to one or more TNF blockers, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter.

Embodiment 30. A method of treating moderate to severe hidradenitis suppurativa (HS) in a patient, wherein the patient has had an inadequate response or intolerance to one or more TNF blockers, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter, wherein the patient achieves one or more of the endpoints selected from the group consisting of: (1) HiSCR 50; (2) Numeric Rating Scale (NRS) 30; (3) HiSCR 75; (4) HiSCR 90; (5) >1.5 reduction from baseline in draining fistula count; (6) >21.3% reduction from baseline in draining fistula count; (7) no increase in draining tunnels (dTs) from baseline; (8) complete elimination of dTs; (9) IHS4-55; (10) IHS4-75; and (11) IHS4-90.

Further benefits of the present disclosure will be apparent to one skilled in the art from reading this patent application. The embodiments of the disclosure described in the following paragraphs are intended to illustrate the invention and should not be deemed to narrow the scope of the invention.

The methods described herein provide an effective and safe dosing regimen for administering lutikizumab to treat hidradenitis suppurativa in a subject. Lutikizumab, a dual-variable domain immunoglobulin (DVD-Ig), is a unique molecule that, unlike currently available inhibitors of the IL-1 pathway, specifically neutralizes both IL-1α and IL-1β without interfering with IL-1RA-mediated regulatory functions in the IL-1 pathway. In a preclinical model of inflammation, blockade of both IL-1α and IL-1β provided better efficacy than inhibition of either cytokine alone (Wu et al. Nat Biotechnol. 2007; 25(11):1290-7). The present disclosure demonstrated that inhibition of both IL-1α and IL-1β by lutikizumab improves the signs and symptoms associated with HS. Given its unique mechanism of action, lutikizumab offers clinical advantages in the treatment of HS, a disease with few approved treatment options and a high unmet need.

### Lutikizumab

Lutikizumab (also referred to as “ABT-981”) is a dual-variable domain immunoglobulin (DVD-Ig) that comprises outer variable domains (VH1 and VL1) that bind human IL-1β and inner variable domains (VH2 and VL2) that bind human IL-1α.

Lutikizumab comprises two identical light chains and two identical heavy chains covalently attached through a full complement of inter- and intra-molecular disulfide bands in a pattern similar to IgG1 antibodies. The heavy and light chain sequences of Lutikizumab are shown in Table 1.

TABLE 1

| | |
|---|---|
| Light | DIQMTQSPSS LSASVGDRVT ITCRASGNIH |
| Chain | NYLTWYQQTP GKAPKLLIYN AKTLADGVPS |
| | RFSGSGSGTD YTFTISSLQP EDIATYYCQH |
| | FWSIPYTFGQ GTKLQITRTV AAPDIQMTQS |
| | PSSVSASVGD RVTITCRASQ GISSWLAWYQ |
| | QKPGKAPKLL IYEASNLETG VPSRFSGSGS |
| | GSDFTLTISS LQPEDFATYY CQQTSSFLLS |
| | FGGGTKVEHK RTVAAPSVFI FPPSDEQLKS |
| | GTASVVCLLN NFYPREAKVQ WKVDNALQSG |
| | NSQESVTEQD SKDSTYSLSS TLTLSKADYE |
| | KHKVYACEVT HQGLSSPVTK SFNRGEC |
| | (SEQ ID NO: 1) |
| Heavy | EVQLVESGGG VVQPGRSLRL SCSASGFIFS |
| Chain | RYDMSWVRQA PGKGLEWVAY ISHGGAGTYY |
| | PDSVKGRFTI SRDNSKNTLF LQMDSLRPED |
| | TGVYFCARGG VTKGYFDVWG QGTPVTVSSA |
| | STKGPQVQLV ESGGGVVQPG RSLRLSCTAS |
| | GFTFSMFGVH WVRQAPGKGL EWVAAVSYDG |
| | SNKYYAESVK GRFTISRDNS KNILFLQMDS |
| | LRLEDTAVYY CARGRPKVVI PAPLAHWGQG |
| | TLVTFSSAST KGPSVFPLAP SSKSTSGGTA |
| | ALGCLVKDYF PEPVTVSWNS GALTSGVHTF |
| | PAVLQSSGLY SLSSVVTVPS SSLGTQTYIC |
| | NVNHKPSNTK VDKKVEPKSC DKTHTCPPCP |
| | APEAAGGPSV FLFPPKPKDT LMISRTPEVT |
| | CVVVDVSHED PEVKFNWYVD GVEVHNAKTK |
| | PREEQYNSTY RVVSVLTVLH QDWLNGKEYK |
| | CKVSNKALPA PIEKTISKAK GQPREPQVYT |
| | LPPSREEMTK NQVSLTCLVK GFYPSDIAVE |
| | WESNGQPENN YKTTPPVLDS DGSFFLYSKL |
| | TVDKSRWQQG NVFSCSVMHE ALHNHYTQKS |
| | LSLSPGK |
| | (SEQ ID NO: 2) |

Light chain and heavy chain variable regions are in bold, and linkers are underlined in Table 1 above.

Additional description of Lutikizumab can be found on pages 291-293 of the WHO Drug Information, Vol. 30, No. 2, 2016, the content of which is incorporated by reference in its entirety.

### Definitions

Where a numeric range is recited herein, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated.

The singular forms “a,” “an” and “the” include plural referents unless the context clearly dictates otherwise.

The term “and/or” as used in a phrase such as “A and/or B” herein is intended to mean “A and B”, “A or B”, “A” or “B”.

The term “about” generally refers to a range of numbers that one of skill in the art would consider equivalent to the recited value (i.e., having the same function or result). In many instances, the term “about” may include numbers that are rounded to the nearest significant figure. In certain instances, the term “about” may be used to denote values falling within ±20% of the recited values, e.g., within ±15%, ±10%, ±7.5%, ±5%, ±4%, ±3%, ±2% or ±1% of the recited values.

The term “baseline” means the first measurement of the targeted variable just before the administration of the studied therapy.

Unless the context requires otherwise, the terms “comprise,” “comprises,” and “comprising” are used on the basis and clear understanding that they are to be interpreted inclusively, rather than exclusively, such that they indicate the inclusion of the recited feature but without excluding one or more other such features.

The term “patient”, “subject”, “individual” and the like refers to humans.

The term “treating” used herein means that the administration of lutikizumab is sufficient to reduce or ameliorate the severity and/or duration of hidradenitis suppurativa (HS), or one or more symptoms thereof.

As used herein, the term “effective amount” or “therapeutically effective amount” refers to the amount of lutikizumab that is sufficient to reduce or ameliorate the severity and/or duration of hidradenitis suppurativa (HS), or one or more symptoms thereof. The therapeutically effective amount of lutikizumab may, for example, reduces the total abscess and inflammatory nodule (AN) count, with no increase in abscess count and no increase in draining fistula-count. The therapeutically effective amount of lutikizumab may, for example, reduces worst skin pain (maximal daily pain), as assessed by the Patient's Global Assessment (GPA) of Skin Pain. In some embodiments, the therapeutically effective amount of lutikizumab may be sufficient for the subject or the patient to achieve one or more of the clinical responses selected from the group consisting of: (1) HiSCR 50; (2) Numeric Rating Scale (NRS) 30; (3) HiSCR 75; (4) HiSCR 90; (5) >1.5 (e.g., >2.5, >3.0, or >4.2) reduction from baseline in draining fistula count; (6) >21.3% (e.g., >34.1%, >44.1%, or >68.3%) reduction from baseline in draining fistula count; (7) no increase in draining tunnels (dTs) from baseline; (8) complete elimination of dTs; (9) IHS4-55; (10) IHS4-75; and (11) IHS4-90.

### Dosing

In one embodiment, the present disclosure relates to a method of treating hidradenitis suppurativa in a subject comprising administering to the subject a therapeutically effective amount of lutikizumab, wherein the therapeutically effective amount of lutikizumab comprises a first dose and a second dose of lutikizumab, wherein the first dose comprises about 300 mg to about 600 mg of lutikizumab, and the second dose comprises about 300 mg of lutikizumab.

The first dose may comprise about 300 mg to about 600 mg of lutikizumab. For example, the first dose of lutikizumab may comprise from about 300 mg to about 550 mg, from about 300 mg to about 500 mg, from about 300 mg to about 450 mg, from about 300 mg to about 400 mg, from about 300 mg to about 350 mg, from about 350 mg to about 600 mg, from about 350 mg to about 550 mg, from about 350 mg to about 500 mg, from about 350 mg to about 450 mg, from about 350 mg to about 400 mg, from about 400 mg to about 600 mg, from about 400 mg to about 550 mg, from about 400 mg to about 500 mg, from about 400 mg to about 450 mg, from about 450 mg to about 600 mg, from about 450 mg to about 550 mg, from about 450 mg to about 500 mg, from about 500 mg to about 600 mg, from about 500 mg to about 550 mg, or from about 550 mg to about 600 mg of lutikizumab.

In some embodiments, the first dose may comprise about 300 mg, about 350 mg, about 400 mg, about 450 mg, about 500 mg, about 550 mg, or about 600 mg of lutikizumab. In certain embodiments, the first dose may comprise about 300 mg of lutikizumab. In certain embodiments, the first dose may comprise about 600 mg of lutikizumab.

In one embodiment, the first dose is administered to the subject at Week 0 (Day 1). In one embodiment, the first dose of about 300 mg to about 600 mg (e.g., 300 mg, 400 mg, 500 mg, or 600 mg) of lutikizumab is administered to the subject at Week 0 (Day 1). For example, in one embodiment, the first dose of about 300 mg of lutikizumab is administered to the subject at Week 0 (Day 1). In another embodiment, the first dose of about 600 mg of lutikizumab is administered to the subject at Week 0 (Day 1).

In one embodiment, the second dose is administered to the subject at Week 1 (Day 8). In one embodiment, the second dose of about 300 mg of lutikizumab is administered to the subject at Week 1 (Day 8).

In one embodiment, the method of treating hidradenitis suppurativa in a subject comprises administering to the subject a therapeutically effective amount of lutikizumab, wherein the therapeutically effective amount of lutikizumab comprises administration of the first dose of lutikizumab at Week 0 (Day 1) and the second dose of lutikizumab at Week 1 (Day 8). In one embodiment, the first dose of about 300 mg to about 600 mg (e.g., 300 mg, 400 mg, 500 mg, or 600 mg) of lutikizumab is administered to the subject at Week 0 (Day 1), and the second dose of about 300 mg of lutikizumab is administered to the subject at Week 1 (Day 8). In one embodiment, the first dose of about 300 mg of lutikizumab is administered to the subject at Week 0 (Day 1), and the second dose of about 300 mg of lutikizumab is administered to the subject at Week 1 (Day 8). In one embodiment, the first dose of about 600 mg of lutikizumab is administered to the subject at Week 0 (Day 1), and the second dose of about 300 mg of lutikizumab is administered to the subject at Week 1 (Day 8).

In one embodiment, the method of treating hidradenitis suppurativa in a subject comprises administering to the subject a therapeutically effective amount of lutikizumab, wherein the therapeutically effective amount of lutikizumab comprises administration of the first dose of about 300 mg to about 600 mg of lutikizumab at Week 0 (Day 1), the second dose of about 300 mg of lutikizumab at Week 1 (Day 8), and about 300 mg of lutikizumab at Week 2 (Day 15) and every week thereafter.

In one embodiment, the method of treating hidradenitis suppurativa in a subject comprises administering to the subject a therapeutically effective amount of lutikizumab, wherein the therapeutically effective amount of lutikizumab comprises administration of the first dose of about 300 mg to about 600 mg of lutikizumab at Week 0 (Day 1), the second dose of about 300 mg of lutikizumab at Week 1 (Day 8), and about 300 mg of lutikizumab at Week 2 (Day 15) or Week 3 (Day 22) and every other week thereafter.

In one embodiment, the method of treating hidradenitis suppurativa in a subject comprises administering to the subject a therapeutically effective amount of lutikizumab, wherein the therapeutically effective amount of lutikizumab comprises administration of the first dose of about 300 mg to about 600 mg of lutikizumab at Week 0 (Day 1), the second dose of about 300 mg of lutikizumab at Week 1 (Day 8), and about 300 mg of lutikizumab every week starting Week 2 (Day 15) for a period of time, and after the period of time, administration of about 300 mg of lutikizumab to the subject every other week.

In one embodiment, the method of treating hidradenitis suppurativa in a subject comprises administering to the subject a therapeutically effective amount of lutikizumab, wherein the therapeutically effective amount of lutikizumab comprises administration of the first dose of about 300 mg to about 600 mg of lutikizumab at Week 0 (Day 1), the second dose of about 300 mg of lutikizumab at Week 1 (Day 8), and about 300 mg of lutikizumab every other week starting Week 2 (Day 15) or Week 3 (Day 22) for a period of time, and after the period of time, administration of about 300 mg of lutikizumab to the subject every week.

The period of time may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 weeks. For example, in one embodiment, the period of time may be 12, 13, 14, 15, or 16 weeks.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every week thereafter.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every other week thereafter.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every week starting Week 2 (Day 15) for a period of time; and (d) after the period of time, administration of about 300 mg of lutikizumab to the subject every other week. The period of time may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 weeks. For example, in one embodiment, the period of time may be 14 weeks.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 300 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every other week starting Week 2 (Day 15) for a period of time; and (d) after the period of time, administration of about 300 mg of lutikizumab to the subject every week. The period of time may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 weeks. For example, in one embodiment, the period of time may be 14 weeks.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) and every week thereafter.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); and (c) administration of about 300 mg of lutikizumab to the subject at Week 2 (Day 15) or Week 3 (Day 22) and every other week thereafter.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every week starting Week 2 (Day 15) for a period of time; and (d) after the period of time, administration of about 300 mg of lutikizumab to the subject every other week. The period of time may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 weeks. For example, in one embodiment, the period of time may be 14 weeks.

In one embodiment, the method of treating hidradenitis suppurativa in a subject comprises administering to the subject a therapeutically effective amount of lutikizumab, wherein the therapeutically effective amount of lutikizumab comprises: (a) administration of the first dose of about 600 mg of lutikizumab to the subject at Week 0 (Day 1); (b) administration of the second dose of about 300 mg of lutikizumab to the subject at Week 1 (Day 8); (c) administration of about 300 mg of lutikizumab to the subject every other week starting Week 2 (Day 15) or Week 3 (Day 22) for a period of time; and (d) after the period of time, administration of about 300 mg of lutikizumab to the subject every week. The period of time may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 weeks. For example, in one embodiment, the period of time may be 13 or 14 weeks.

Additional exemplary doses and dose regimens are shown in Table 2 below.

TABLE 2
Doses and Dose Regimens

| | | Additional | |
|---|---|---|---|
| First dose | Second dose | doses | |
| (mg) | (mg) | (mg) | Frequency of additional doses |
| 300 Week | 300 Week 1 | 300 | Week 2 (Day 15) and EW thereafter |
| 0 (Day 1) | (Day 8) | | |
| 300 Week | 300 Week 1 | 300 | Week 2 (Day 15) and EW thereafter for a |
| 0 (Day 1) | (Day 8) | | period of time; then EOW |
| 300 Week | 300 Week 1 | 300 | Week 2 (Day 15) and EOW thereafter |
| 0 (Day 1) | (Day 8) | | |
| 300 Week | 300 Week 1 | 300 | Week 2 (Day 15) and EOW thereafter for |
| 0 (Day 1) | (Day 8) | | a period of time; then EW |
| 600 Week | 300 Week 1 | 300 | Week 2 (Day 15) and EW thereafter |
| 0 (Day 1) | (Day 8) | | |
| 600 Week | 300 Week 1 | 300 | Week 2 (Day 15) and EW thereafter for a |
| 0 (Day 1) | (Day 8) | | period of time; then EOW |
| 600 Week | 300 Week 1 | 300 | Week 2 (Day 15) or Week 3 (Day 22) and |
| 0 (Day 1) | (Day 8) | | EOW thereafter |
| 600 Week | 300 Week 1 | 300 | Week 2 (Day 15) or Week 3 (Day 22) and |
| 0 (Day 1) | (Day 8) | | EOW thereafter for a period of time; then |
| | | | EW |

EW: Every Week;
EOW: Every Other Week

In one embodiment, at least one dose of lutikizumab is administered via subcutaneous injection (SC). In one embodiment, the first and/or the second doses of lutikizumab are administered via subcutaneous injection (SC). In one embodiment, all doses of lutikizumab are administered via subcutaneous injection (SC).

In one embodiment, provided herein is a method of treating moderate to severe hidradenitis suppurativa (HS) in a patient, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter.

In one embodiment, provided herein is a method of treating moderate to severe hidradenitis suppurativa (HS) in a patient, wherein the patient has had an inadequate response or intolerance to one or more TNF blockers, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter.

In one embodiment, provided herein is a method of treating moderate to severe hidradenitis suppurativa (HS) in a patient, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter, wherein the patient achieves one or more of the endpoints selected from the group consisting of: (1) HiSCR 50; (2) Numeric Rating Scale (NRS) 30; (3) HiSCR 75; (4) HiSCR 90; (5) >1.5 (e.g., >2.5, >3.0, or >4.2) reduction from baseline in draining fistula count; (6) >21.3% (e.g., >34.1%, >44.1%, or >68.3%) reduction from baseline in draining fistula count; (7) no increase in draining tunnels (dTs) from baseline; (8) complete elimination of dTs; (9) IHS4-55; (10) IHS4-75; and (11) IHS4-90.

In one embodiment, provided herein is a method of treating moderate to severe hidradenitis suppurativa (HS) in a patient, wherein the patient has had an inadequate response or intolerance to one or more TNF blockers, comprising administration to the patient a therapeutically effective amount of lutikizumab, wherein the therapeutically effective amount of lutikizumab comprises subcutaneous administration of an initial dose of 600 mg at Week 0 (Day 1), followed by subcutaneous administration of 300 mg at Week 1 (Day 8), and subcutaneous administration of 300 mg once every week thereafter, wherein the patient achieves one or more of the endpoints selected from the group consisting of: (1) HiSCR 50; (2) Numeric Rating Scale (NRS) 30; (3) HiSCR 75; (4) HiSCR 90; (5) >1.5 (e.g., >2.5, >3.0, or >4.2) reduction from baseline in draining fistula count; (6) >21.3% (e.g., >34.1%, >44.1%, or >68.3%) reduction from baseline in draining fistula count; (7) no increase in draining tunnels (dTs) from baseline; (8) complete elimination of dTs; (9) IHS4-55; (10) IHS4-75; and (11) IHS4-90.

In one embodiment, at least one dose of lutikizumab is delivered by multiple injections. In one embodiment, all doses of lutikizumab are delivered by multiple injections. In one embodiment, the multiple injections comprise at least two (e.g., 2, 3, 4, 5, or 6) injections. For example, in one embodiment, the first dose or the initial dose of 600 mg at Week 0 (Day 1) is delivered by four of 150 mg injections. In one embodiment, the multiple injections may be spread over a course of time, e.g., a course of a 1-hour period, 3-hour period, 6-hour period, 12-hour period, 18-hour period, 24-hour period, 30-hour period, 36-hour period, 42-hour period, 48-hour period, 3-day period, 4-day period, 5-day period, 6-day period, or 7-day period.

### Patient Selection

In one embodiment, the methods described in the present disclosure are used to treat a subject afflicted with moderate to severe hidradenitis suppurativa.

In one embodiment, the subject treated with the methods described herein is an adult (≥18 years of age) patient. In one embodiment, the subject treated with the methods described herein is an adult or adolescent (≥16 years of age) patient.

In one embodiment, the subject treated with the methods described herein have had a diagnosis of moderate to severe hidradenitis suppurativa for at least 6 months prior to baseline (e.g., as determined through medical history and interview of the subject).

In one embodiment, the methods described in the present disclosure are used to treat a subject who has one or more of the following: (1) a total abscess and inflammatory nodule (AN) count of ≥5; (2) HS lesions present in at least two distinct anatomic areas at baseline; (3) at least 1 anatomic area of HS involvement characterized as Hurley Stage II or higher at baseline; and (4) draining fistula count of ≤20 at baseline. In one embodiment, the methods described in the present disclosure are used to treat a subject who has all of the following: (1) a total abscess and inflammatory nodule (AN) count of ≥5; (2) HS lesions present in at least two distinct anatomic areas at baseline; (3) at least 1 anatomic area of HS involvement characterized as Hurley Stage II or higher at baseline; and (4) draining fistula count of ≤20 at baseline.

In one embodiment, the subject has not received any prior biologic therapy, i.e., is naïve to biologic therapy (“bio-naïve”) for HS. In one embodiment, the subject has not had prior exposure to anti-IL-1 or anti-IL-1 type 1 receptor biologic agents (e.g., anakinra, canakinumab, rilonacept, or bermekimab).

In one embodiment, the subject treated with the methods described herein has been treated with a prior biologic therapy for HS and had inadequate response to this therapy (“bio-IR”). For example, in one embodiment, the subject failed one or more anti-TNF treatments for hidradenitis suppurativa. The failure to one or more anti-TNF treatments may be defined as one of the following: inadequate response or loss of response despite at least 12 weeks of therapy with at least one TNF antagonists, or intolerance to at least one TNF antagonists. Accordingly, in one embodiment, the subject treated with the methods described herein has inadequate response or loss of response despite at least 12 weeks of therapy with at least one TNF antagonists, or intolerance to at least one TNF antagonists.

### Clinical Responses

In one embodiment, the method of treating hidradenitis suppurativa in a subject further comprises measuring at least one clinical response (or clinical endpoint) of the subject after one or more doses of lutikizumab (e.g., after the first or the second dose of lutikizumab) is administered. In one embodiment, clinical responses are measured one week after each dose of lutikizumab is administered. In certain embodiments, clinical responses are measured one week after the last dose of lutikizumab is administered. In certain embodiments, clinical responses are measured one week after 1, 2, 3, 4, 5, 6, 7, 8, 9, 10, 11, 12, 13, 14, 15, or 16 doses of lutikizumab (e.g., after the first or the second dose of lutikizumab) are administered.

The clinical responses (clinical endpoints) may comprise: (1) HiSCR 50 (defined as at least a 50% reduction from baseline in the total abscess and inflammatory nodule (AN) count, with no increase in abscess count and no increase in draining fistula-count relative to baseline); (2) at least a 30% reduction and at least 1-unit reduction from baseline in worst skin pain (maximal daily pain), as assessed by the Patient's Global Assessment (GPA) of Skin Pain (Numeric Rating Scale [NRS]30) among subjects with baseline NRS≥3; (3) HiSCR 75 (defined as at least a 75% reduction from baseline in the total AN count, with no increase in abscess count and no increase in draining fistula-count relative to baseline); (4) HiSCR 90 (defined as at least a 90% reduction from baseline in the total AN count, with no increase in abscess count and no increase in draining fistula-count relative to baseline); (5) >1.5 (e.g., >2.5, >3.0, or >4.2) reduction from baseline in draining fistula count; (6) >21.3% (e.g., >34.1%, >44.1%, or >68.3%) reduction from baseline in draining fistula count; (7) no increase in draining tunnels (dTs) from baseline; (8) complete elimination of dTs; (9) IHS4-55 (defined as at least a 55% reduction from baseline in International Hidradenitis Suppurativa Severity Score System (IHS4) [Number of nodules (inflammatory)×1]+[Number of abscesses×2]+[Number of draining tunnels×4]); (10) IHS4-75 (defined as at least a 75% reduction from baseline in IHS4 [Number of nodules (inflammatory)×1]+[Number of abscesses×2]+[Number of draining tunnels×4]); and/or (11) IHS4-90 (defined as at least a 90% reduction from baseline in IHS4 [Number of nodules (inflammatory)×1]+[Number of abscesses×2]+[Number of draining tunnels×4]).

The subject may achieve one or more of the above clinical endpoints after administration of the therapeutically effective amount of lutikizumab. In one embodiment, the subject achieves HiSCR 50 after administration of the therapeutically effective amount of lutikizumab. In another embodiment, the subject achieves HiSCR 75 after administration of the therapeutically effective amount of lutikizumab. In another embodiment, the subject achieves HiSCR 90 after administration of the therapeutically effective amount of lutikizumab. In another embodiment, the subject achieves NRS30 after administration of the therapeutically effective amount of lutikizumab. In another embodiment, the subject achieves >1.5 (e.g., >2.5, >3.0, or >4.2) reduction from baseline in draining fistula count after administration of the therapeutically effective amount of lutikizumab. In another embodiment, the subject achieves >21.3% (e.g., >34.1%, >44.1%, or >68.3%) reduction from baseline in draining fistula count after administration of the therapeutically effective amount of lutikizumab. In another embodiment, the subject achieves no increase in draining tunnels (dTs) from baseline after administration of the therapeutically effective amount of lutikizumab. In another embodiment, the subject achieves complete elimination of dTs after administration of the therapeutically effective amount of lutikizumab. In another embodiment, the subject achieves IHS4-55 after administration of the therapeutically effective amount of lutikizumab. In another embodiment, the subject achieves IHS4-75 after administration of the therapeutically effective amount of lutikizumab. In another embodiment, the subject achieves IHS4-90 after administration of the therapeutically effective amount of lutikizumab. In one embodiment, the subject achieves one or more of the above clinical endpoints after administration of at least one dose (e.g., 1, 2, 3, 4, 5, 6, 7, 8, 9, 10, 11, 12, 13, 14, 15, or 16 doses) of lutikizumab. In one embodiment, the subject achieves one or more of the above clinical endpoints after administration of 9 doses of lutikizumab. In another embodiment, the subject achieves one or more of the above clinical endpoints after administration of 16 doses of lutikizumab.

### Example 1: Phase II Study M20-262

A phase 2, randomized, double-blind, parallel-group, placebo-controlled, dose-ranging, multicenter study (M20-262) was carried out to evaluate the efficacy and safety of lutikizumab (Luti) in adult subjects with moderate to severe Hidradenitis Suppurativa (HS) who have failed anti-TNF therapy.

Objective(s): The study assessed the safety and efficacy of lutikizumab 300 mg every week (EW), 300 mg every other week (EOW), and 100 mg EOW versus placebo for the treatment of signs and symptoms of moderate to severe HS in adult subjects who have failed anti-TNF therapy.

Study Population and Number of Subjects Enrolled: A total of 153 subjects with moderate to severe HS who had failed anti-TNF therapy were randomized. 129 subjects completed the study drug, and 24 subjects discontinued the study drug for various reasons. Most patients (70.6 percent) had severe baseline Hurley Stage 3 disease—the most extensive form of HS—characterized by scarring, lesions and sinus tracts.

Key Eligibility Criteria: Subjects were at least ≥18 years of age at Screening with a clinical diagnosis of HS for at least 1 year prior to Baseline (as determined by the investigator). Subjects had a total AN count of ≥5 at Baseline and had the presence of HS lesions in at least 2 distinct anatomic areas. Subjects had also failed anti-TNF treatment for HS defined as one of the following: inadequate response or loss of response (in the opinion of the investigator) despite at least 12 weeks of therapy with ≥1 TNF antagonists or intolerance to ≥1 TNF antagonists. Demonstration of intolerance to TNF antagonists required no minimal duration of use.

### Study Design:

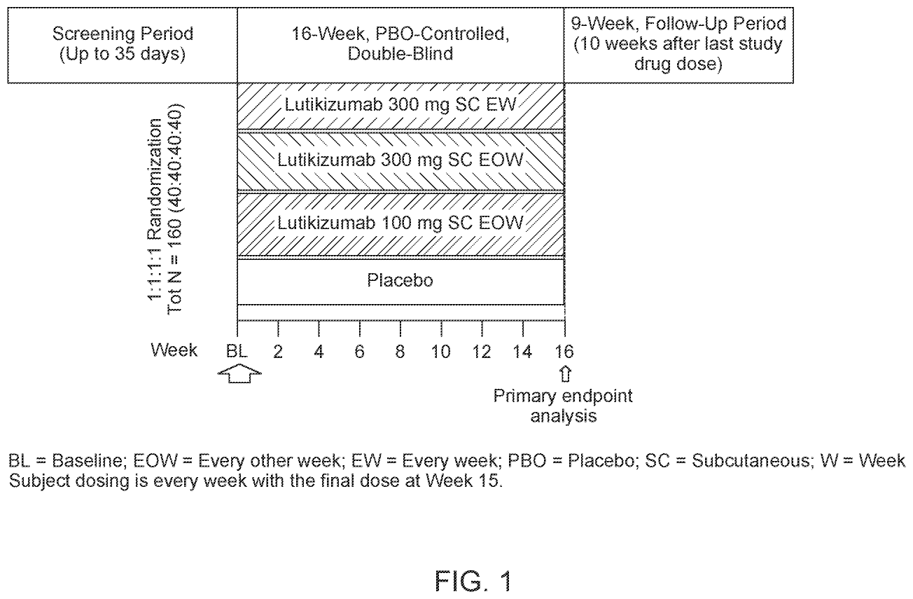

As shown in FIG. 1, the study comprised a 35-day Screening Period, a 16-week double-blind placebo-controlled Treatment Period, and a 9-week Follow-Up Period (10 weeks after the last dose of study drug [administered on Week 15]).

Subjects who met all eligibility criteria were centrally randomized at Baseline in a 1:1:1:1 ratio to one of 4 treatment groups (Planned N=40 per group), stratified by the worst Hurley Stage across all affected anatomic regions (<3 or 3) at Baseline:
Group 1 (Luti 300 mg EW): Lutikizumab 300 mg subcutaneous (SC) EW
Group 2 (Luti 300 mg EOW): Lutikizumab 300 mg SC EOW (loading dose of 300 mg SC at Baseline and Week 1, followed by 300 mg SC at Week 2 and EOW thereafter)
Group 3 (Luti 100 mg EOW): Lutikizumab 100 mg SC EOW (loading dose of 100 mg SC at Baseline and Week 1, followed by 100 mg SC at Week 2 and EOW thereafter)
Group 4 (PBO): Placebo SC EW

Subjects in Group 1 were dosed with 300 mg lutikizumab at Baseline and Weeks 1, 2, 3, 4, 5, 6, 7, 8, 9, 10, 11, 12, 13, 14, and 15.

Subjects in Group 2 were dosed with 300 mg lutikizumab at Baseline and Week 1, followed by 300 mg lutikizumab at Weeks 2, 4, 6, 8, 10, 12, and 14, and placebo at Weeks 3, 5, 7, 9, 11, 13, and 15.

Subjects in Group 3 were dosed with 100 mg lutikizumab at Baseline and Week 1, followed by 100 mg lutikizumab at Weeks 2, 4, 6, 8, 10, 12, and 14, and placebo at Weeks 3, 5, 7, 9, 11, 13, and 15.

Subjects in Group 4 were given matching placebo at Baseline and Weeks 1, 2, 3, 4, 5, 6, 7, 8, 9, 10, 11, 12, 13, 14, and 15.

The primary efficacy objective was to assess a higher rate of achieving HiSCR 50 at Week 16 with each lutikizumab group compared to PBO in adult subjects who have failed anti-TNF therapy.

Endpoint(s): The primary endpoint was the achievement of Hidradenitis Suppurativa Clinical Response (HiSCR) 50 at Week 16. HiSCR 50 is defined as at least a 50% reduction from Baseline in the total abscess and inflammatory nodule (AN) count, with no increase in abscess count and no increase in draining fistula-count relative to Baseline.

The secondary endpoint was the achievement of at least a 30% reduction and at least 1-unit reduction from Baseline in worst skin pain (maximal daily pain) at Week 16, as assessed by the Patient's Global Assessment (PGA) of Skin Pain (Numeric Rating Scale [NRS]30) among subjects with baseline NRS≥3.

Additional endpoints were also evaluated to assess the depth of response of lutikizumab compared to the placebo. For example, achievement of HiSCR75, HiSCR90 and the International Hidradenitis Suppurativa Severity Score System (IHS4) endpoints, IHS4-55, IHS4-75, and IHS4-90, that dynamically assess draining fistulas/tunnels in addition to nodules and abscesses was also evaluated at Week 16. Achievement of HiSCR 75 and HiSCR 90 was defined as ≥75% and ≥90% reduction, respectively, in the total AN count with no increase in abscess count and no increase in draining fistula count relative to baseline. Achievement of IHS4-55, IHS4-75, and IHS4-90 was defined as at least a 55%, 75%, or 90% reduction, respectively, from baseline in IHS4 [Number of nodules (inflammatory)×1]+[Number of abscesses×2]+[Number of draining tunnels×4].

### Example 2: Phase II Study M20-262 Results

The final efficacy and safety results (including data from the safety follow-up Period) from the Phase II M20-262 study were reported as follows.

### Efficacy:

Luti 300 mg EOW and EW showed high response rates with 98.5% and 89.3% probability of being better than placebo, respectively. Pre-specified analysis showed a higher response rate in the Luti 300 mg EOW group than the 300 mg EW group. Driven by the observed difference between Luti 300 mg EOW and Luti 300 mg EW at Week 3 (while subjects from both groups received the same doses before the assessment), a post-hoc analysis adjusting for important baseline factors estimated a similar response rate in the Luti 300 mg EOW and EW groups.

Both Luti 300 mg EOW and EW doses also showed greater response rates over PBO for the secondary endpoint and selected additional endpoints at Week 16 (details in Table 3). Comparing the 300 mg EOW and EW group: EOW group outperformed EW group in the pre-specified analysis of HiSCR 75; EW group outperformed EOW group in the post-hoc baseline-adjusted analysis of HiSCR 75, and the high stringency endpoint of HiSCR 90 (in both pre-specified and baseline-adjusted analyses).

### Primary and Secondary Endpoints:

Both Luti 300 mg EOW and 300 mg EW showed greater response rates over PBO in the primary endpoint, HiSCR 50 at Week 16, with a posterior probability of observing a positive treatment difference versus PBO of 98.5% and 89.3%, respectively. A sensitivity analysis with the Cochran-Mantel-Haenszel (CMH) test showed consistent results (Table 3).

Greater efficacy of Luti 300 mg EOW and Luti 300 mg EW were also observed in the secondary endpoint and additional endpoints at Week 16 (Table 3).

TABLE 3
Week 16 Efficacy Results

| | | Resp (%); Treatment Diff Δ vs. PBO [95% CI] | | |
|---|---|---|---|---|
| | Resp (%) | Luti 100 mg | Luti 300 mg | |
| Endpoint | PBO | EOW | EOW | Luti 300 mg EW |
| (all at Week 16) | N = 40 | N = 37 | N = 37 | N = 39 |

Primary Endpoint: HiSCR 50²

| | | | | |
|---|---|---|---|---|
| Pre-specified | 35.0 | 27.0 | 59.5 | 48.7 |
| Bayesian Approach | | Δ: −7.7 [−27.6, | Δ: 23.8 [2.2, 44.3] | Δ: 13.4 [−7.8, |
| | | 12.7] | | 34.0] |
| Pre-specified CMH⁴ | 35.0 | 27.0 | 59.5 | 48.7 |
| | | Δ −8.0 [−23.2, 5.6] | Δ 24.5 [9.1, 39.5] | Δ 13.7 [−1.3, |
| | | | | 28.8] |
| Baseline-adjusted¹ | 38.6 | 33.7 | 51.1 | 51.0 |
| | | Δ: −4.8 [−26.3, | Δ: 12.5 [−9.6, | Δ: 12.4, [−9.4, |
| | | 16.6] | 34.6] | 34.2] |

Secondary Endpoint: Skin Pain NRS30³

| | | | | |
|---|---|---|---|---|
| Pre-specified | N = | N = 27 | N = 29 | N = 23 |
| Bayesian Approach | 31 12.9 | 22.2 | 34.5 | 34.8 |
| | | Δ: 9.1 [−10.2, | Δ: 20.9 [0.3, 41.5] | Δ: 21.3 [−0.7, |
| | | 29.0] | | 43.8] |

Selected Additional Endpoint: HiSCR 75²

| | | | | |
|---|---|---|---|---|
| Pre-specified | 17.5 | 16.2 | 45.9 | 38.5 |
| Bayesian Approach | | Δ: −1.1 [−17.9, | Δ: 27.8 [7.9, 46.9] | Δ: 20.5 [1.4, 39.3] |
| | | 15.7] | | |
| Baseline-adjusted¹ | 18.9 | 20.5 | 37.5 | 47.2 |
| | | Δ: 1.6 [−16.2, | Δ: 18.6 [−1.1, | Δ: 28.3 [8.5, |
| | | 19.4] | 38.4] | 48.11 |

Selected Additional Endpoint: HiSCR 90²

| | | | | |
|---|---|---|---|---|
| Pre-specified | 5.0 | 8.1 | 13.5 | 25.6 |
| Bayesian Approach | | Δ: 3.1 [−8.4, 15.5] | Δ: 8.4 [−4.4, 22.4] | Δ: 20.2 [5.4, 35.9] |
| Baseline-adjusted¹ | 6.3 | 10.1 | 13.9 | 31.4 |
| | | Δ: 3.8 [−8.5, 16.1] | Δ: 7.6 [−5.9, 21.0] | Δ: 25.1 [8.7, |
| | | | | 41.5] |

¹Post-hoc analysis adjusting for baseline factors: current tobacco use (Yes/No [former or never]), reason for discontinuing prior anti-TNF due to loss of response (Yes/No), draining fistula count, skin pain NRS, hsCRP.
²HiSCR 50/75/90: at least a 50%/75%/90% reduction from Baseline in the total AN count (sum of abscess and inflammatory nodule counts), with no increase in abscess count and no increase in draining fistula count.
³Skin Pain NRS30: at least a 30% and at least 1-unit reduction from Baseline in skin pain NRS (Numeric Rating Scale) among subjects with Baseline NRS ≥ 3.
⁴Cochran-Mantel-Haenszel test.

Phase 2 results show that adults with moderate to severe hidradenitis suppurativa (HS) who had previously failed anti-TNF therapy who received lutikizumab (ABT-981) 300 mg every other week or 300 mg weekly achieved higher response rates (59.5 percent, nominal p=0.027 and 48.7 percent, nominal p=0.197, respectively) than placebo (35.0 percent) in the primary endpoint of achieving HS Clinical Response (HiSCR 50) at week 16 (Table 4).

In addition to achieving higher response rates in the primary endpoint and despite most patients having severe disease, the trial also showed patients receiving lutikizumab 300 mg weekly and 300 mg every other week achieved higher rates of improved skin pain via NRS3 and HiSCR75, a higher threshold of HS clinical response, compared to placebo (Table 4).

TABLE 4
Results from select endpoints are as follows:

| | | | Response (%); Treatment Diff vs. PBO^{#} | | |
|---|---|---|---|---|---|
| | | Response | P-value⁺ | | |
| | | (%) | Luti 100 mg | Luti 300 mg | Luti 300 mg |
| Endpoints | | PBO | EOW | EOW | EW |
| (All at Week 16) | | (N = 40) | (N = 37) | (N = 37) | (N = 39) |
| Primary | HiSCR50 | 35.0 | 27.0 | 59.5 | 48.7 |
| | | | Δ: −9.7 | Δ: 24.1 | Δ: 13.8 |
| | | | p = 0.345 | p = 0.027 | p = 0.197 |
| Secondary | Skin | N = 31 | N = 27 | N = 29 | N = 23 |
| | Pain | 12.9 | 22.2 | 34.5 | 34.8 |
| | NRS30* | | Δ: 9.4 | Δ: 21.8 | Δ: 19.8 |
| | | | p = 0.330 | p = 0.039 | p = 0.066 |
| Additional | HiSCR75 | 17.5 | 16.2 | 45.9 | 38.5 |
| | | | Δ: −2.2 | Δ: 28.2 | Δ: 21.0 |
| | | | p = 0.795 | p = 0.005 | p = 0.031 |

^{#}Cochran-Mantel-Haenszel test adjusted for the stratification factor (Baseline Hurley Stage <3 and 3)
⁺All p values are nominal
*Analyzed among patients with baseline Skin Pain NRS ≥ 3

Phase 2 results also show that patients treated with lutikizumab 300 mg EOW and 300 mg EW showed greater improvement in draining fistula count at week 16 than those treated with placebo (Table 5).

TABLE 5

| | | Lutikizumab | Lutikizumab | Lutikizumab |
|---|---|---|---|---|
| | | 100 mg | 300 mg | 300 mg |
| Parameter, LS mean; | Placebo | EOW | EOW | EW |
| Difference vs. Placebo | (N = 33) | (N = 28) | (N = 32) | (N = 35) |
| Change from baseline* | | | | |
| Draining fistula count | −1.5 | −2.5 | −4.2 | −3.0 |
| | | Δ −1.0 | Δ −2.6 | Δ −1.5 |
| | | P = .374 | P = .013 | P = .162 |
| Percent change from baseline* | (N = 16) | (N = 22) | (N = 21) | (N = 22) |
| (among patients with | | | | |
| baseline ≥ 3) | | | | |
| Draining fistula count | −21.3 | −34.1 | −68.3 | −44.1 |
| | | Δ −12.7 | Δ −47.0 | Δ −22.8 |
| | | P = .523 | P = .025 | P = .253 |

*Reported by MMRM, the mixed-effect model repeat measures analysis with treatment, visit, treatment by visit interaction, stratification factor (baseline worst Hurley Stage) and baseline measurement in the model.
Baseline is defined as the last non-missing value prior to the first dose of study drug or randomization if no study drug was given.
Patients with non-missing baseline and at least one post-baseline value is included in the analyses.
EOW, every other week; EW, every week; PBO, placebo

Achievement of complete elimination or no increase in draining tunnels (dTs) was also evaluated. A greater proportion of patients (NRI-MI) receiving lutikizumab 300 mg EW and 300 mg EOW achieved no increase in dTs from baseline (79.5%, 83.8%, respectively) versus placebo (65.0%) and complete elimination of dTs (28.2%, 29.7%, respectively) versus placebo (17.5%) at week 16. Thus, in this hard-to-treat HS patient population, treatment with lutikizumab showed greater improvement in dTs than placebo.

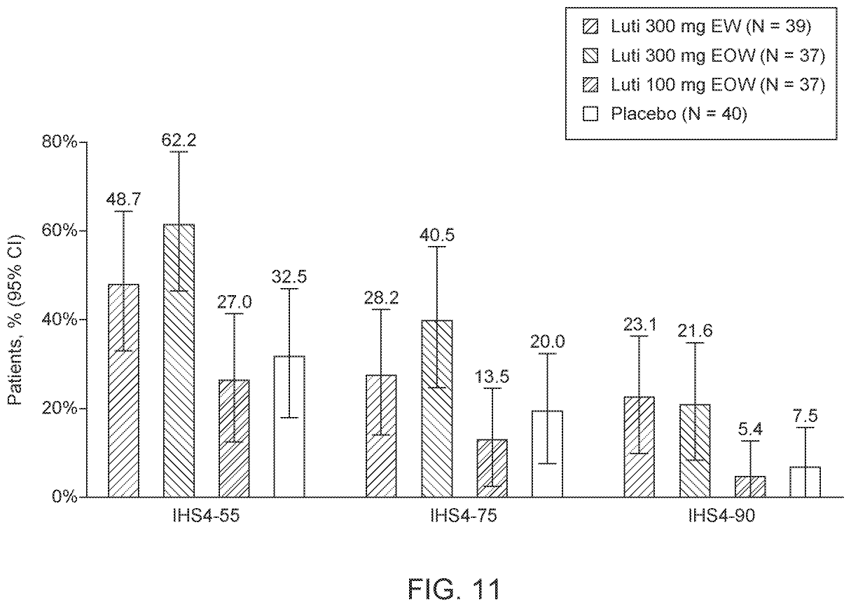

Patients treated with lutikizumab 300 mg EOW and 300 mg EW also experienced higher response rates in IHS4-55, IHS4-75, and IHS4-90 at week 16 than those treated with placebo (FIG. 11).

### Efficacy Over Time

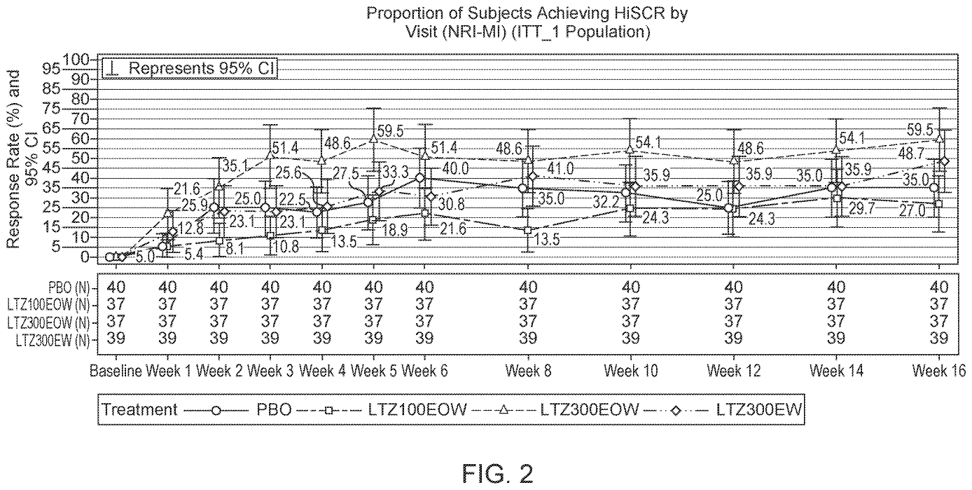

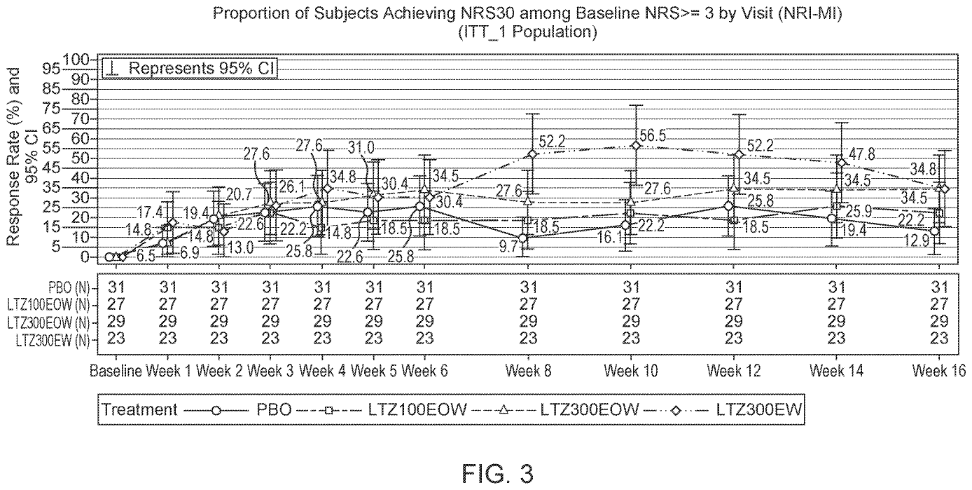

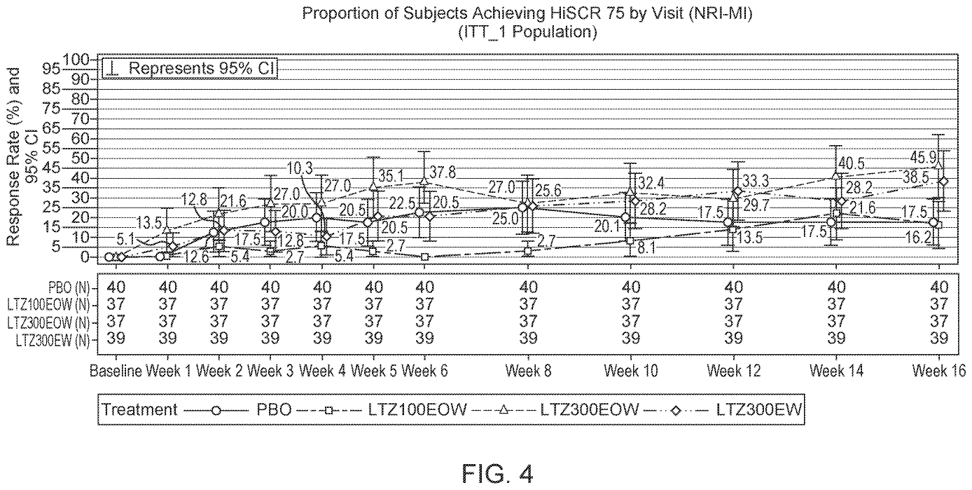

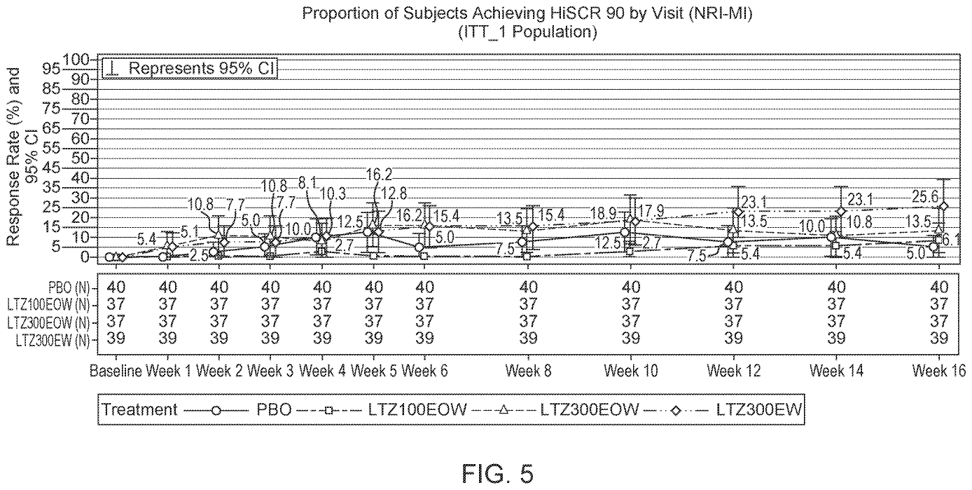

Response rates and the 95% confidence intervals (CIs) in HiSCR 50, skin pain NRS30 among Baseline NRS≥3, HiSCR 75, and HiSCR 90, at each visit, based on pre-specified analysis, are presented in FIG. 2, FIG. 3, FIG. 4, and FIG. 5, respectively.

### Baseline-Adjusted Efficacy Analysis

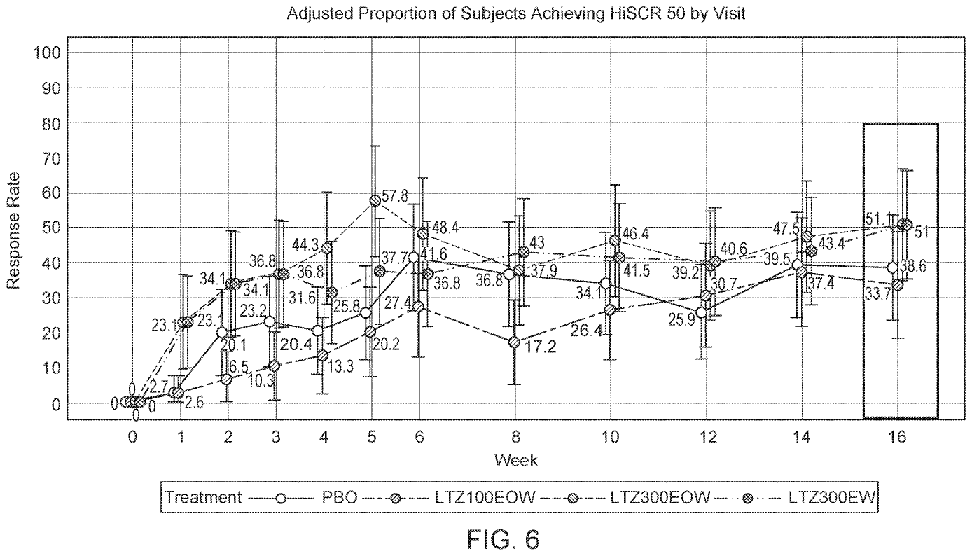

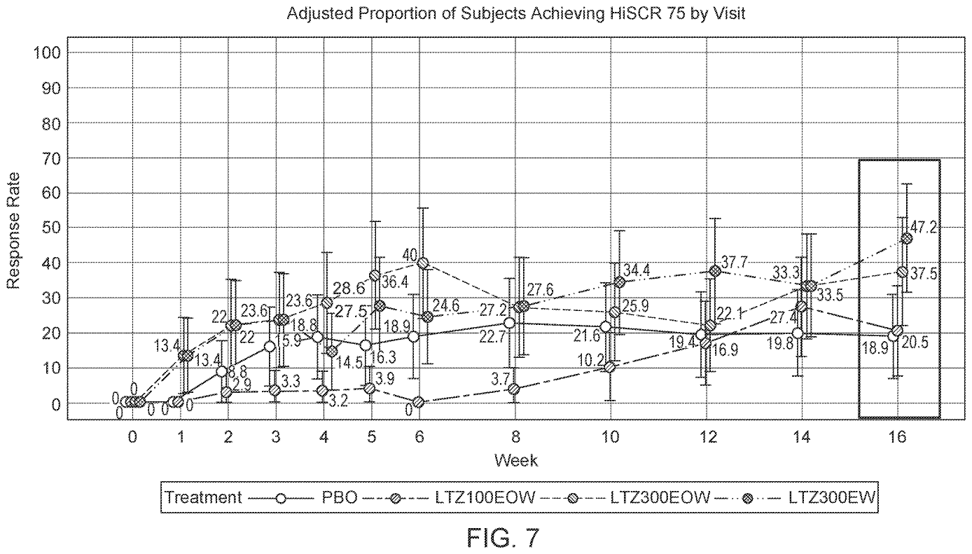

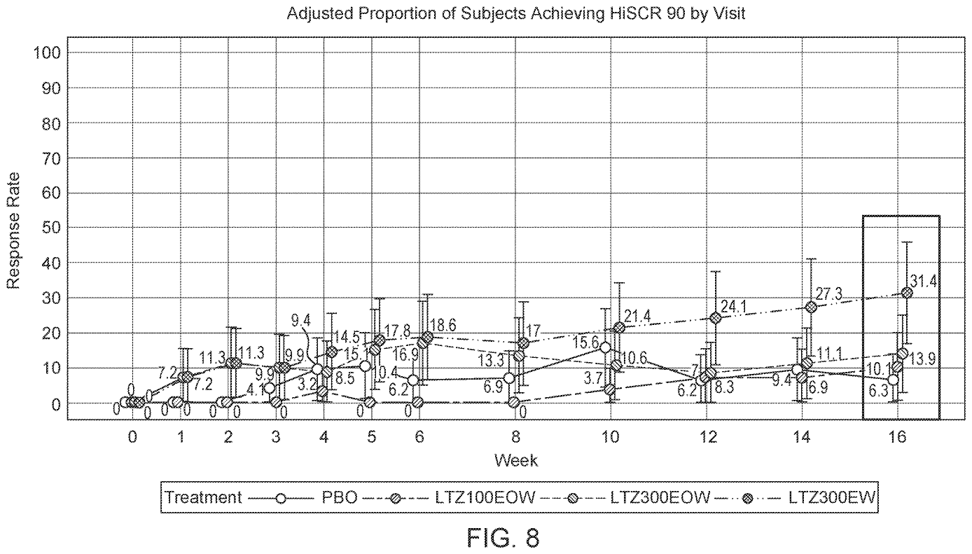

A post-hoc analysis was performed. After adjusting for important baseline covariates, the estimated Week 16 efficacy of Luti 300 mg EW was similar to Luti 300 mg EOW for HiSCR50 (FIG. 6), and higher than Luti 300 mg EOW for HiSCR 75 (FIG. 7) and HiSCR 90 (FIG. 8), supporting HiSCR75 as the primary endpoint for Phase 3 study. The baseline covariates were identified by LASSO (Least Absolute Shrinkage and Selection Operator) (Tibshirani (1996) 58(1):267-288).

### Safety:

All doses were generally safe and well-tolerated. No events of neutropenia were reported, and no grade 3 or 4 laboratory evaluations of neutropenia were observed. There were no dose-dependent trends in any adverse events (AEs), serious adverse events (SAEs), infections, or serious infections.

### Conclusion:

Both Luti 300 mg EOW and Luti 300 mg EW showed greater efficacy over placebo for the primary endpoint, the second endpoint, and selected additional endpoints for the treatment of HS in the TNF-IR population. All doses were generally safe and well-tolerated with no dose-related treatment-emergent adverse events (TEAEs).

### Example 3: Phase III Study M20-465

A Phase 3, global, randomized, double-blinded, placebo-controlled, multicenter study to evaluate efficacy and safety of Lutikizumab 300 mg SC every week after a loading dose of 600 mg SC at Baseline relative to placebo in adult and adolescent subjects ages 16 and older with moderate to severe HS is conducted. Subjects naïve to biologic therapy (bio-naïve) for HS and those who have been treated with a prior biologic therapy for HS and had inadequate response to this therapy (bio-IR) are eligible. Enrollment of Bio-naive subjects is at most 70%.

Objectives: For Period 1, the primary objective is to assess the efficacy and safety of Lutikizumab 300 mg every week versus placebo in all corners for the treatment of signs and symptoms of moderate to severe hidradenitis suppurativa (HS) in subjects ages 16 and older.

For Period 2, the primary objectives are: (1) to assess the efficacy and safety of continuing Lutikizumab 300 mg EW or switching to Lutikizumab 300 mg EOW in subjects who received Lutikizumab in Period 1; and (2) to evaluate the benefit of switching to Lutikizumab in subjects who received placebo in Period 1.

Number of Subjects: Approximately 1280 subjects are enrolled and randomized in a 1:1 ratio to one of the 2 treatment arms (approximately 640 subjects in the active treatment arm and approximately 640 subjects in the placebo arm).

### Eligibility Criteria:

1. Subjects must be ≥18 or ≥16 (in countries where permitted) years of age at the Screening Visit.
2. Subjects must have a diagnosis of moderate to severe HS for at least 6 months prior to Baseline as determined by the investigator (i.e., through medical history and interview of subject).
3. Subjects must have a total AN count of ≥5 at Baseline.
4. Subjects must have HS lesions present in at least two distinct anatomic areas at Baseline.
5. At least 1 anatomic area of HS involvement characterized as Hurley Stage II or higher at Baseline.
6. Subjects must have a draining fistula count of ≤20 at Baseline.
7. Subjects must not have a history of active skin disease other than HS that could interfere with the assessment of HS, including skin infections (e.g., bacterial, fungal, or viral) requiring systemic treatment within 4 weeks of the Baseline visit.
8. Subjects must have laboratory values meeting the following criteria within the screening period (prior to the first dose of study drug): serum aspartate transaminase (AST)≤2×upper limit of normal (ULN); serum alanine transaminase (ALT)≤2×ULN; serum total bilirubin≤2.0 mg/dL, except for subjects with isolated elevation of indirect bilirubin relating to Gilbert syndrome; total white blood cell (WBC) count≥3,000/μL; absolute neutrophil count (ANC)≥2,000/μL; platelet count≥100,000/μL; and hemoglobin≥9.0 g/dL.
9. Subjects must not have any evidence of: hepatitis B virus (HBV) or hepatitis C virus (HCV) infection. HBV infection is defined as: Hepatitis B surface antigen (HBs Ag) positive (+) test or detected sensitivity on the HBV DNA polymerase chain reaction (PCR) qualitative test for subjects who are hepatitis B core antibody (HBc Ab) positive (+) (and for hepatitis B surface antibody [HBs Ab] positive [+] subjects where mandated by local requirements). HCV infection is defined as: HCV RNA detectable in any subject with anti-HCV antibody (HCV Ab).
10. Subjects must not have a confirmed positive anti-HIV antibody (HIV Ab) test.
11. Subjects must not have active TB or meet TB exclusionary parameters.
12. Subjects must not have any evidence of active systemic infection or clinically important infection during the 2 weeks prior to Baseline as assessed by the investigator.
13. Subjects must not have a history of an organ transplant which requires continued immunosuppression.
14. Subjects must not have a history of malignancy within the last 5 years except for successfully treated non-melanoma skin cancer (NMSC) or localized carcinoma in situ of the cervix.
15. Subjects must not have a history of drug or alcohol abuse within the last 6 months.
16. Subjects must not have a history of an allergic reaction or significant sensitivity to constituents of the study drug (and its excipients) and/or other products in the same class.
17. Subjects must not have had any major surgery performed within 12 weeks prior to Baseline or planned surgeries during the conduct of the study (e.g., hip replacement, aneurysm removal, stomach ligation).
18. Subjects must not have a history of or concurrent clinically significant medical conditions, such as recent [within last 6 months] myocardial infarction, cerebrovascular accident, or any other reason, including any physical, psychological, or psychiatric condition that in the opinion of the Investigator would compromise the safety or interfere with the subject's participation in this study or would make the subject an unsuitable candidate to receive study drug or would put the subject at risk by participating in the protocol.
19. Subjects are judged to be in good general health, as determined by the Principal Investigator based upon the results of a medical history, physical examination, laboratory profile, and a 12-lead electrocardiogram (ECG) performed during the Screening period.
20. Female subjects of childbearing potential must practice at least 1 protocol specified method of birth control, which is effective from study day 1 through at least 10 weeks after the last dose of study drug (local practices may require 2 methods of birth control). Female subjects of non-childbearing potential do not need to use birth control.
21. All female subjects of child-bearing potential must have a negative serum pregnancy test at the Screening Visit and a negative urine pregnancy test at Baseline prior to the first dose of study drug. All female subjects of child-bearing potential must not be pregnant, breastfeeding, or considering becoming pregnant during the study or for approximately 10 weeks after the last dose of study drug.
22. Subjects must not have had prior exposure to anti-IL-1 or anti-IL-1 type 1 receptor biologic agents (e.g., anakinra, canakinumab, rilonacept, bermekimab).
23. Subjects must have discontinued biologic therapies with immunosuppressive potential prior to the first dose of study drug as specified in the washout procedures, or at least five times the mean terminal elimination half-life of the medication prior to the first dose of study drug. Specified washout periods are as follows; if not specified below biologic therapies must be discontinued at least 5 times the mean terminal elimination half-life of drug or 3 months prior to Baseline, whichever is longer: ≥4 weeks for etanercept; ≥8 weeks for adalimumab, infliximab, certolizumab, golimumab, abatacept, tocilizumab, and ixekizumab; ≥16 weeks for secukinumab; ≥12 weeks for ustekinumab, guselkumab, and Risankizumab.
24. Subjects must not have had previous treatment with any cell-depleting therapies including but not limited to anti-CD20 (e.g., rituximab) within 12 months prior to Baseline or until B cell count returns to normal level.
25. Subjects must not have used prescription topical therapies (including topical antibiotics) that can also be used to treat HS within 14 days prior to Baseline visit.
26. Subjects must not have received systemic, non-antibiotic, non-biologic therapies (e.g., JAK inhibitor, methotrexate) that can also be used to treat HS within 4 weeks prior to Baseline visit. Subjects taking metformin for non-HS indications must be on a documented stable dose and dosing regimen for at least 28 days prior to the Baseline visit and may not adjust their regimen until after Week 52 assessments are completed.
27. Subjects must not have received any systemic (including oral) antibiotic treatment for HS or any other inflammatory disorder within 14 days prior to Baseline visit.
28. Subjects must be willing to and are required to use a daily antiseptic wash on their HS lesions starting at least 14 days prior to Baseline. Allowable antiseptic washes are limited to one of the following: chlorhexidine gluconate, benzoyl peroxide, benzalkonium chloride, benzethonium chloride, or dilute bleach in bathwater.
29. Subjects who are on non-opioid analgesics, including acetaminophen (paracetamol) or ibuprofen for HS or non-HS related pain, must be on a stable dose and dosing regimen for at least 14 days preceding the Baseline visit and are expected to remain stable at least until the Week 16 visit. Otherwise, subjects must not have received oral analgesics, including but not limited to opioids, antiepileptics (e.g., gabapentin), tricyclics (e.g., nortriptyline), or selective serotonin norepinephrine reuptake inhibitors (e.g., duloxetine, venlafaxine) within 14 days prior to the Baseline visit. See Concomitant Medication Section for permissible analgesic therapy for HS-related pain.
30. Subjects must not have received any live vaccine within 4 weeks prior to the first dose of study drug (Baseline) or have an expected need for live vaccination during study participation including at least 10 weeks after the last dose of study drug. JYNNEOS vaccine (live, attenuated, non-replicating vaccine) may be administered.
31. Subjects must not have been treated with any other investigational drug within 30 days or 5 half-lives of the drug (whichever is longer) prior to the first dose of study drug or must not be currently enrolled in another clinical study.

### Study Design:

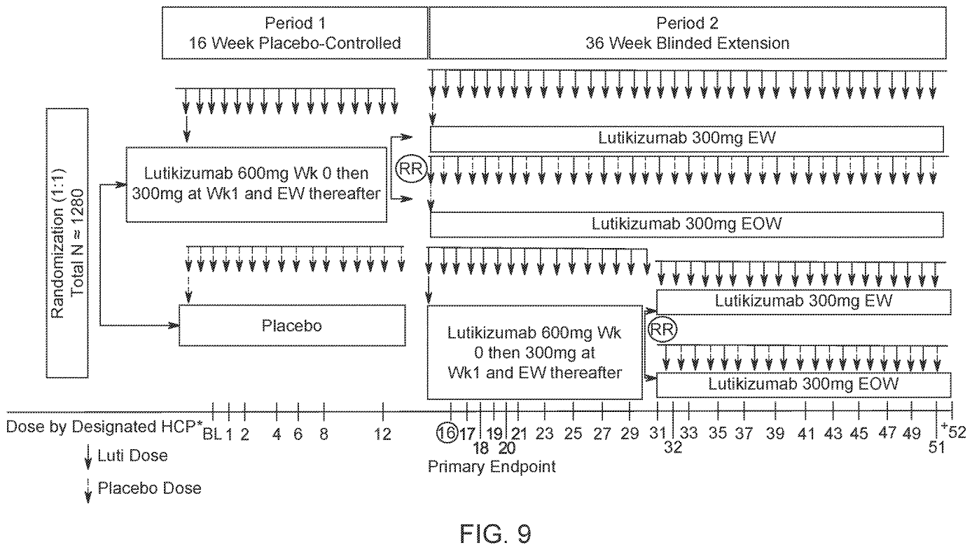

As shown in FIG. 9, the study comprises a 35-day Screening Period, a 16-week double-blinded placebo-controlled treatment period (Period 1), a 36-week double-dummy extension treatment period (Period 2), and a 10-week Follow Up Period after last study drug dosing with an option to enter a separate long term extension study.

### Period 1

During Period 1, subjects who meet all eligibility criteria are randomized in a 1:1 ratio to one of two treatment groups:
(1) Lutikizumab: Loading dose of 600 mg Lutikizumab SC at baseline (BL), followed by 300 mg Lutikizumab SC at week 1 and EW thereafter through week 16 (last dose in Period 1 is at Week 15).
(2) Placebo

### Period 2

During Period 2, subjects assigned to the Lutikizumab treatment arm in Period 1 who complete Week 16 of the study are re-randomized in a 1:1 ratio (stratified by HiSCR 75 at Week 16) to one of two treatment groups:
(1) Lutikizumab 300 mg SC EW starting at Week 16 through Week 52 (last dose in Period 2 is at Week 51).
(2) Lutikizumab 300 mg SC EOW starting at Week 16 through Week 52 (last dose in Period 2 is at Week 51).

Subjects assigned to the placebo arm in Period 1 who complete week 16 of the study initiate Lutikizumab with a loading dose of 600 mg SC at Week 16 followed by 300 mg SC at week 17 and EW thereafter up to Week 31. Starting at week 32, these subjects are re-randomized in a 1:1 ratio (stratified by HiSCR 75 at Week 32) to one of two treatment groups:
(1) Lutikizumab 300 mg SC EW starting at Week 32 through Week 52 (last dose in Period 2 is at Week 51).
(2) Lutikizumab 300 mg SC EOW starting at Week 32 through Week 52 (last dose in Period 2 is at Week 51).

All subjects who complete week 52 of the pivotal study are eligible to enroll into the Long-Term Extension Study.

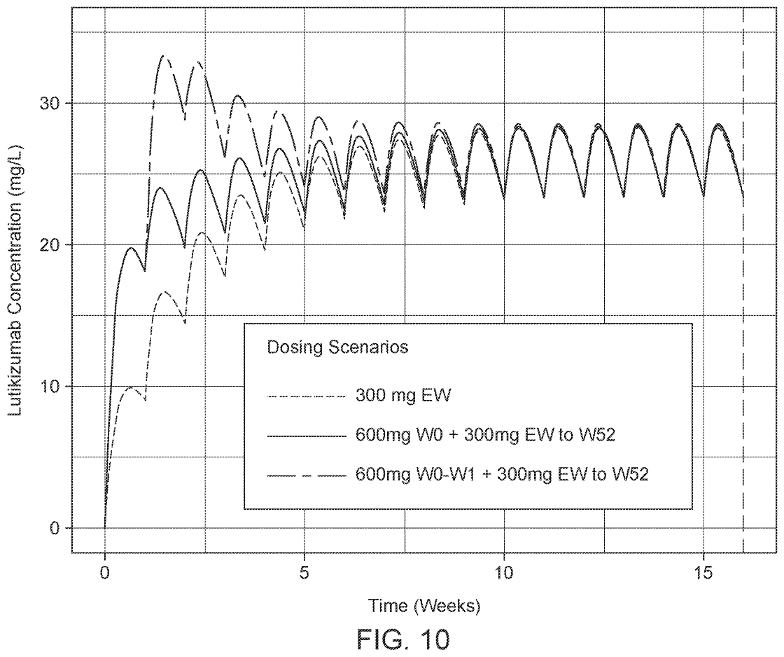

Dose Rationale: A 600 mg loading dose is administered at Week 0 to provide faster onset to achieve lutikizumab steady state exposure without exceeding the maximum exposure already safely tested in HS (FIG. 10).

Efficacy Endpoints: The primary efficacy endpoint is the achievement of Hidradenitis Suppurativa Clinical Response 75 (HiSCR 75) at Week 16. HiSCR 75 is defined as at least a 75% reduction from baseline in the total abscess and inflammatory nodule (AN) count, with no increase in abscess count and no increase in draining fistula count relative to Baseline.

The secondary efficacy endpoints include: (1) achievement of NRS30 (at least a 30% reduction and at least 2-unit reduction from Baseline in Numerical Rating Scale [NRS] in the Patient's Global Assessment of HS-related skin pain) at Week 8 among patients with NRS≥3 at Baseline. NRS30 is based on worst skin pain in a 24-hour recall period (maximal daily pain); (2) change from Baseline in Draining Fistula at Week 16; (3) change from Baseline in Hidradenitis Suppurativa Impact Assessment (HSIA) Emotional Domain at Week 16; (4) change from Baseline in Hidradenitis Suppurative Impact Assessment (HSIA) at Week 16; (5) change from Baseline in Hidradenitis Suppurativa Symptom Assessment (HSSA) Worst Drainage Score at Week 16; (6) change from Baseline in Dermatology Life Quality Index (DLQI) for adult patients at Week 16; (7) achievement of HiSCR 90 (at least a 90% reduction in the total AN count with no increase in abscess count and no increase in draining fistula count relative to Baseline) at Week 16; (8) change from Baseline in Hidradenitis Suppurativa Impact Assessment (HSIA) Mobility Domain at Week 16; (9) change from Baseline in HSSA at Week 16; (10) change from Baseline in HS-related odor (smell), based on HSSA Question 8, at Week 16; (11) achievement of HiSCR 50 (at least a 50% reduction in the total AN count with no increase in abscess count and no increase in draining fistula count relative to Baseline) at Week 16; and (12) incidence of HS flare, defined as at least one occurrence of a ≥25% increase in AN count with a minimum absolute increase of 2 relative to Baseline during the first 16 weeks (Period 1).

All variables listed as primary and ranked secondary efficacy endpoints above are also analyzed at all scheduled visits they are assessed, in addition to those listed above.

Additionally, the following endpoints are evaluated at scheduled visits the assessments are measured: (1) percent change from Baseline in AN count; (2) percent change from Baseline in draining fistula count; (3) change from Baseline in AN count; (4) change from Baseline in International Hidradenitis Suppurativa Severity Score System (IHS4) score; (5) change from baseline in Numerical Rating Scale [NRS] in the Patient's Global Assessment of HS-related skin pain; (6) achievement of IHS4-55 (at least a 55% reduction in the IHS4 score relative to Baseline); (7) achievement of “much improved” or “very much improved” on the Total Patient Global Impression of Change (PGIC—HSSA Total); (8) achievement of ≥1 grade improvement in Total Patient Global Impression of Severity relative to Baseline (PGIS—HSSA Total); (9) change from Baseline in Euro-QoL 5 Dimensions 5 Levels Health State (EQ-5D-5L); (10) change from Baseline in Work Productivity and Impairment (WPAI); (11) change from Baseline in Patient-Reported Outcomes Measurement Information System (PROMIS) Short-Form v1.0—Sleep Disturbance 4a for adult subjects or PROMIS Pediatric SF4 v1.0 Sleep Disturbance for adolescent subjects ages 16 to 17 years old; (12) change from Baseline in Hospital Anxiety and Depression Scale (HADS) Depression Subscale; (13) change from Baseline in HADS Anxiety Subscale; and (14) change from Baseline in Children's DLQI (CDLQI) for adolescent patients 16 to 17 years of age.

Safety Endpoints: Treatment emergent adverse events (TEAEs), serious adverse events (SAEs), adverse events (AEs) of special interest (AESI), AEs leading to discontinuation are assessed. Vital signs, laboratory tests, ECG, and physical examination findings are recorded.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1.-30. (canceled)

31. A method of treating hidradenitis suppurativa (HS) in a subject, comprising administering to the subject a therapeutically effective amount of lutikizumab, wherein the therapeutically effective amount of lutikizumab comprises:
(1) a first dose of lutikizumab comprising about 300 mg to about 600 mg of lutikizumab, wherein the first dose is administered to the subject at Week 0 (Day 1),
(2) a second dose of lutikizumab comprising about 300 mg of lutikizumab, wherein the second dose is administered to the subject at Week 1 (Day 8), and
(3) one or more further doses of lutikizumab, each comprising about 300 mg of lutikizumab, wherein the one or more further doses are administered to the subject at: (a) Week 2 (Day 15) and every week or every other week thereafter for a period of time; or (b) Week 3 (Day 22) and every other week thereafter for a period of time.

32. The method of claim 31, wherein the first dose of lutikizumab comprises about 600 mg of lutikizumab.

33. The method of claim 31, wherein the first dose of lutikizumab comprises about 300 mg of lutikizumab.

34. The method of claim 31, wherein the first dose of lutikizumab comprises about 600 mg of lutikizumab and the one or more further doses of lutikizumab are administered to the subject at Week 2 (Day 15) and every week thereafter.

35. The method of claim 31, wherein the first dose of lutikizumab comprises about 600 mg of lutikizumab and the one or more further doses of lutikizumab are administered to the subject at Week 2 (Day 15) and every other week thereafter.

36. The method of claim 31, wherein the first dose of lutikizumab comprises about 600 mg of lutikizumab and the one or more further doses of lutikizumab are administered to the subject at Week 3 (Day 22) and every other week thereafter.

37. The method of claim 31, wherein the first dose of lutikizumab comprises about 300 mg of lutikizumab and the one or more further doses of lutikizumab are administered to the subject at Week 2 (Day 15) and every week thereafter.

38. The method of claim 31, wherein the first dose of lutikizumab comprises about 300 mg of lutikizumab and the one or more further doses of lutikizumab are administered to the subject at Week 2 (Day 15) and every other week thereafter.

39. The method of claim 31, wherein the period of time is 12, 13, 14, 15, or 16 weeks.

40. The method of claim 31, wherein, after the period of time, a dose comprising about 300 mg of lutikizumab is administered every week to the subject.

41. The method of claim 31, wherein, after the period of time, a dose comprising about 300 mg of lutikizumab is administered every other week to the subject.

42. A method of treating hidradenitis suppurativa (HS) in a subject, comprising administering to the subject a therapeutically effective amount of lutikizumab, wherein the therapeutically effective amount of lutikizunab comprises a dose comprising about 300 mg of lutikizunab administered every week or every other week to the subject.

43. A method of treating hidradenitis suppurativa (HS) in a population of subjects, comprising administering to each subject in the population:
(1) a first dose of lutikizunab comprising about 300 mg to about 600 mg of lutikizumab, wherein the first dose is administered to each subject at Week 0 (Day 1);
(2) a second dose of lutikizunab comprising about 300 mg of lutikizumab, wherein the second dose is administered to each subject at Week 1 (Day 8); and
(3) one or more further doses of lutikizumab, each comprising about 300 mg of lutikizumab, wherein the one or more further doses are administered to each subject at: (a) Week 2 (Day 15) and every week or every other week thereafter for a period of time; or (b) Week 3 (Day 22) and every other week thereafter for a period of time,
wherein the severity and/or duration of hidradenitis suppurativa (HS) or one or more symptoms thereof is reduced or ameliorated in a greater proportion of the population of subjects as compared to a population of subjects administered with a placebo.

44. A method of treating hidradenitis suppurativa (HS) in a subject, comprising administering to the subject a therapeutically effective amount of lutikizumab, wherein the therapeutically effective amount of lutikizumab comprises:
(1) a first dose of lutikizumab comprising about 600 mg of lutikizumab, wherein the first dose is administered to the subject at Week 0 (Day 1);
(2) a second dose of lutikizumab comprising about 300 mg of lutikizumab, wherein the second dose is administered to the subject at Week 1 (Day 8); and
(3) one or more further doses of lutikizumab, each comprising about 300 mg of lutikizumab, wherein the one or more further doses are administered to the subject at Week 2 (Day 15) and every week thereafter for a period of time.

45. The method of claim 44, wherein the severity and/or duration of hidradenitis suppurativa (HS) or one or more symptoms thereof is reduced or ameliorated in the subject.

46. The method of claim 44, wherein the subject achieves (1) reduced total abscess and inflammatory nodule (AN) count as compared to baseline, with no increase in abscess count and no increase in draining fistula-count; and/or (2) reduced worst skin pain (maximal daily pain) as compared to baseline, as assessed by the Patient's Global Assessment (GPA) of Skin Pain.

47. The method of claim 44, wherein the subject achieves one or more endpoints selected from the group consisting of:
(1) Hidradenitis Suppurativa Clinical Response (HiSCR) 50;
(2) NRS30 (at least a 30% reduction and at least 1-unit reduction from baseline in Numerical Rating Scale (NRS) in the Patients Global Assessment (GPA) of HS-related skin pain);
(3) HiSCR 75;
(4) HiSCR 90;
(5) >1.5 reduction from baseline in draining fistula count;
(6) >21.3% reduction from baseline in draining fistula count;
(7) no increase in draining tunnels (dTs) from baseline; and
(8) complete elimination of dTs.

48. The method of claim 44, wherein the subject achieves one or more endpoints selected from the group consisting of:
(1) IHS4-55;
(2) IHS4-75; and
(3) IHS4-90.

49. The method of claim 44, wherein the subject achieves HiSCR 50.

50. The method of claim 44, wherein the subject achieves HiSCR 75.

51. The method of claim 44, wherein the subject achieves HiSCR 90.

52. The method of claim 44, wherein the subject has a baseline Numerical Rating Scale (NRS) equal to or greater than 3, and wherein the subject achieves at least a 30% reduction and at least 1-unit reduction from baseline in worst skin pain (maximal daily pain) by Week 16, as assessed by the Patient's Global Assessment (PGA) of Skin Pain.

53. The method of claim 44, wherein the period of time is 12, 13, 14, 15, or 16 weeks.

54. The method of claim 44, wherein the period of time is 14 weeks, and wherein the severity and/or duration of hidradenitis suppurativa (HS), or one or more symptoms thereof is reduced or ameliorated by Week 16.

55. The method of claim 44, wherein, after the period of time, one or more doses of lutikizumab, each comprising 300 mg of lutikizumab, is administered every week to the subject.

56. The method of claim 44, wherein, after the period of time, one or more doses of lutikizumab, each comprising 300 mg of lutikizumab, is administered every other week to the subject.

57. The method of claim 44, wherein each dose of lutikizumab is administered via subcutaneous injection.

58. The method of claim 44, wherein the HS is moderate to severe HS.

59. The method of claim 44, wherein the subject has (a) a total abscess and inflammatory nodule (AN) count of ≥5 at baseline and (b) HS lesions present in at least 2 distinct anatomic areas at baseline.

60. The method of claim 44, wherein the subject is an adult patient or an adolescent patient.

61. The method of claim 44, wherein the subject is naive to a biologic therapy for HS.

62. The method of claim 44, wherein the subject has previously been treated with a biologic therapy for HS and had intolerance or an inadequate response to the biologic therapy.

63. The method of claim 62, wherein the biologic therapy comprises an anti-TNF treatment for HS.
